(19) United States
(12) Patent Application Publication
(10) Pub. No.: US 2012/0123420 A1
(43) Pub. Date: May 17, 2012

(54) POSITIONING GUIDE AND BONE CUTTING GUIDE SYSTEM

(76) Inventor: John Robert Honiball, Western Cape (ZA)

(21) Appl. No.: 13/381,057

(22) PCT Filed: Jun. 24, 2010
(86) PCT No.: PCT/IB10/52899

§ 371 Date: Dec. 27, 2011

(30) Foreign Application Priority Data
Jun. 24, 2009 (ZA) 2009/04421

(51) Int. Cl. A61B 17/56

(52) U.S. Cl. 606/87

(57) ABSTRACT

A bone cutting guide system (110) includes a bone cutting guide assembly (111) and a positioning guide (114). The bone cutting guide assembly (111) includes a base pin (112) and a guide mounting arrangement comprising a placement pin guide (113). The pin (112) has an elongate cylindrical shaft (115) having a penetrating end (117). The positioning guide (114) includes a moulding (116) and a hollow cylindrical guide post (118) fixed to the moulding (116). The moulding (116) is constructed from anatomical data obtained by means of a radiographic scan of the head (55) of the femur (14) prior to surgery. The placement pin guide (113) has a handle (121) and a hollow cylindrical guide sleeve (119) dimensioned to slidingly receive the guide pin (112) therein. In use, the moulding (116) is fitted to the head (55) of the femur. The guiding formation (119) of the pin placement guide (113) is received in the post (118) for guiding the insertion of the pin (112) into the head (55) of the femur (14).

### FIELD OF INVENTION

This invention relates to a positioning guide. This invention relates also to a bone cutting guide system including the positioning guide, for use in guiding the cutting of a predetermined joint bone of a human patient during a joint replacement surgical procedure during which a prosthetic component is fitted to the joint. In this specification the term “predetermined joint bone” of a human patient shall be interpreted to mean a joint bone selected from the group comprising: an acetabular bone, a head of a femur bone, a head of a humerus bone, and a glenoid bone.

### SUMMARY OF INVENTION

According to a first aspect of the invention, there is provided a positioning guide for use with a bone cutting guide assembly for use in a joint replacement surgical procedure for guiding the cutting of at least one prosthetic joint locating face in a region of a predetermined joint bone of a human patient, from which a portion of bone is to be removed, thereby to allow for the secure fitment of a prosthetic joint to said bone in a predetermined orientation which approximates the anatomical normality of said joint bone, the bone cutting guide assembly including a bone cutting guide having at least one cutter guide formation for guiding a cutter for cutting said prosthetic joint locating face in said region of said joint bone; and guide mounting means to which the bone cutting guide is releasably mounted for releasably mounting the bone cutting guide to said joint bone,

the positioning guide including a bone mounting structure in the form of a moulding which is constructed from anatomical data obtained of said region of said joint bone so as to define complementary locating formations which correspond to anatomical formations of said region of said joint bone, thereby to provide for the secure fitment of the bone mounting structure to said region of said joint bone, the bone mounting structure having at least one cutting guide locating formation to which the bone cutting guide of the bone cutting guide assembly is releasably mounted, in use, for positioning the bone cutting guide, relative to said joint bone in an arrangement wherein the cutter guide formation of the bone cutting guide is located in a predetermined position relative to said joint bone so as to facilitate the cutting of said prosthetic joint locating face in said joint bone thereby to provide for the fitment of the prosthetic joint thereto.

The cutting guide locating formation may be in the form of a guide passage defined through the moulding.

The cutting guide locating formation may be in the form of an attachment post projecting from an external side of the moulding.

According to a second aspect of the invention, there is provided a bone cutting guide system for use in a joint replacement surgical procedure for guiding the cutting of at least one prosthetic joint locating face in a region of a predetermined joint bone of a human patient, from which a portion of bone is to be removed, thereby to allow for the secure fitment of a prosthetic joint to said bone in a predetermined orientation which approximates the anatomical normality of said joint bone, the bone cutting guide system including:

a bone cutting guide assembly including:
a) a bone cutting guide having at least one cutter guide formation for guiding a cutter for cutting said prosthetic joint locating face in said region of said bone; and
b) guide mounting means to which the bone cutting guide is releasably mounted for releasably mounting the bone cutting guide to said joint bone; and
   the positioning guide as claimed in Claim 1 for mounting the bone cutting guide assembly to said bone, with the bone cutting guide being fixedly secured to said bone after the mounting of the bone cutting guide to the cutting guide locating formation, with the cutting guide locating formation, the guide mounting means and the bone cutting guide being configured to permit removal of the guide mounting means and the positioning guide while the bone cutting guide remains fixedly secured to the bone, thereby to ensure the undisturbed attachment of the bone cutting guide to the bone at the predetermined position relative to the bone when the positioning guide and the guide mounting means are removed from the bone.

The bone cutting guide may be in the form of an elongate base pin having a penetrating end configured to be imbedded into the bone, in use.

The cutting guide locating formation of the positioning guide may be in the form of an attachment post extending from an external side of the moulding.

The guide mounting means of the bone cutting guide assembly may be in the form of a pin placement guide for guiding the placement of the base pin into the patient's bone, the pin placement guide having a mounting formation for mounting the pin placement guide to the attachment post and at least one pin mounting formation for guiding the insertion of the elongate base pin into the patient's bone.

The attachment post and the mounting formation of the pin placement guide define complementary configurations so as to permit the pin placement guide to be rotatably mounted to the attachment post, to permit the pin mounting formation of the pin placement guide to revolve around the attachment post to permit the selection of the most appropriate location for inserting the base pin into the patient's bone.

The attachment post and the mounting formation of the pin placement guide may both have an angular configuration thereby to permit the pin placement guide to be mounted to the attachment post in a predetermined orientation, thereby to fix the position at which the pin mounting formation of the pin placement guide is located when the attachment post mounting formation of the pin placement guide is mounted to the attachment post.

### BRIEF DESCRIPTION OF THE DRAWINGS

Further features of the invention are described hereinafter by way of a non-limiting example of the invention, with reference to and as illustrated in the accompanying diagrammatic drawings. In the drawings:

FIG. 1 shows a fragmentary perspective view of the bones of a human leg in their anatomically normal position;

FIG. 2A shows a front view of a human pelvis including the hip joints;

FIG. 2B shows a top view of the pelvis of FIG. 2A;

FIG. 2C shows a fragmentary side view of the pelvis of FIG. 2A;

FIG. 2D shows a fragmentary perspective view of a hip bone of the pelvis of FIG. 2A;

FIG. 3A shows a fragmentary perspective view of a glenoid bone;

FIG. 3B shows a fragmentary perspective view of a humerus bone;

FIG. 4 shows a fragmentary perspective view of the hip bone of FIG. 2D wherein the acetabulum has been cut to fit an acetabular component of a hip prosthesis thereto;

FIG. 5 shows a fragmentary perspective view of the head of the femur of the human leg of FIG. 1, which has been cut to fit a femoral component of a hip prosthesis thereto;

FIG. 6A shows a fragmentary perspective view of the glenoid bone of FIG. 3A, which has been cut to fit a glenoid component of a shoulder prosthesis thereto;

FIG. 6B shows a fragmentary perspective view of the humerus of FIG. 3B which has been cut to fit a humerus component of a shoulder prosthesis thereto;

FIG. 7A shows a fragmentary perspective view of the femur of FIG. 5 to which a femoral component of a hip joint prosthesis has been fitted;

FIG. 7B shows a fragmentary side view of the humerus component of the shoulder prosthesis connected to the humerus of FIG. 6B;

FIG. 8 shows a fragmentary view of the acetabum of FIG. 4, to which the acetabular component of the prior art hip prosthesis has been connected;

FIG. 9 shows a fragmentary perspective view of the glenoid bone of FIG. 6A to which the glenoid component of the shoulder prosthesis is connected;

FIGS. 10A to 10G show, in sequence, the various steps in the cutting of the head of the femur during a prior art hip replacement procedure;

FIGS. 11A and 11B illustrate the use of guide tools for reaming the acetabulum of the hip bone of FIG. 2D during a prior art hip replacement surgical procedure;

FIGS. 11C to 11E illustrate the procedure for reaming the acetabulum of the hip bone of FIG. 2D during a prior art hip replacement surgical procedure;

FIG. 12 shows a first embodiment of a bone cutting guide system in accordance with the first aspect of the invention, connected to the femur of FIG. 1;

FIGS. 13A to 13G show a second embodiment of a bone cutting guide system in accordance with the first aspect of the invention, illustrating the use of the bone cutting guide system, in sequence;

FIGS. 14A to 14E show a third embodiment of a bone cutting guide system in accordance with the first aspect of the invention, and illustrating the use of the bone cutting guide system, in sequence;

FIGS. 15A to 15O show a fourth embodiment of a bone cutting guide system in accordance with the first aspect of the invention, illustrating the use of the bone cutting guide system, in sequence; and

FIGS. 16A to 16J show a fifth embodiment of a bone cutting guide system in accordance with the invention, illustrating the use of the bone cutting guide system, in sequence.

### DETAILED DESCRIPTION OF DRAWINGS

The present invention relates to a positioning guide for use with a bone cutting guide assembly and to a bone cutting guide system including the positioning guide, for use in guiding the cutting of a predetermined joint bone of a human patient during a joint replacement surgical procedure.

The patient's articulating joint may require replacement due to injury or deterioration caused by aging, or certain debilitating conditions, such as, for example, arthritis. Joints which may be replaced in this manner, include, but are not limited to knee joints, shoulder joints and hip joints. An anatomically normal hip joint and shoulder joint will be described below.

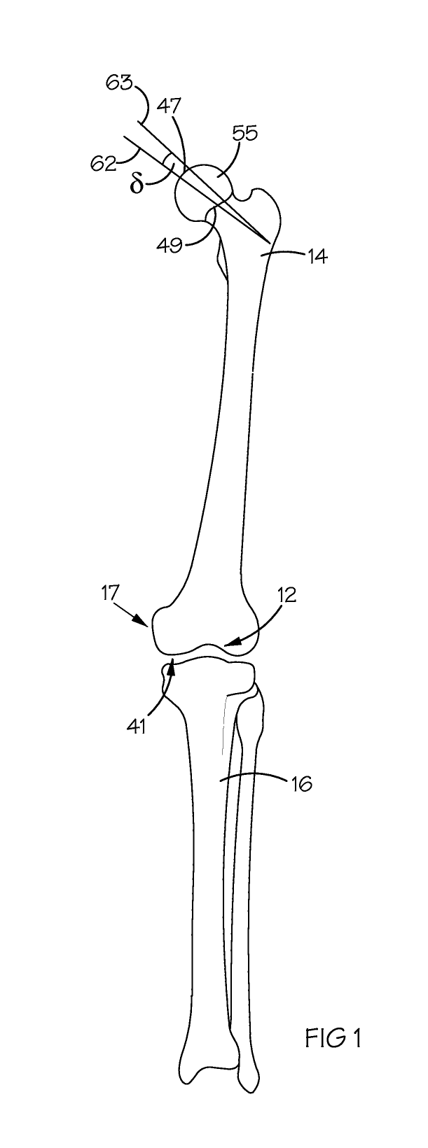

FIG. 1 shows a perspective view of the bones of a human leg in their anatomically normal position. The bones include a femur 14 and a tibia 16, shown in their normal position.

The femur 14 has a lower extremity 12 at its distal end 41 and a femoral head 55 and a neck 49 at its proximal end 47. The femur 14 defines a neutral axis 62 which extends through the centre of the head 55 of the femur 14 and through a central axis of the neck 49 of the femur 14. The neutral axis 62 is known as valgus neutral. The head 55 of the femur 14 also defines an implant axis 63 which is angularly offset from the neutral axis 62 by an angle δ as shown in the drawings, the significance of which will be explained below.

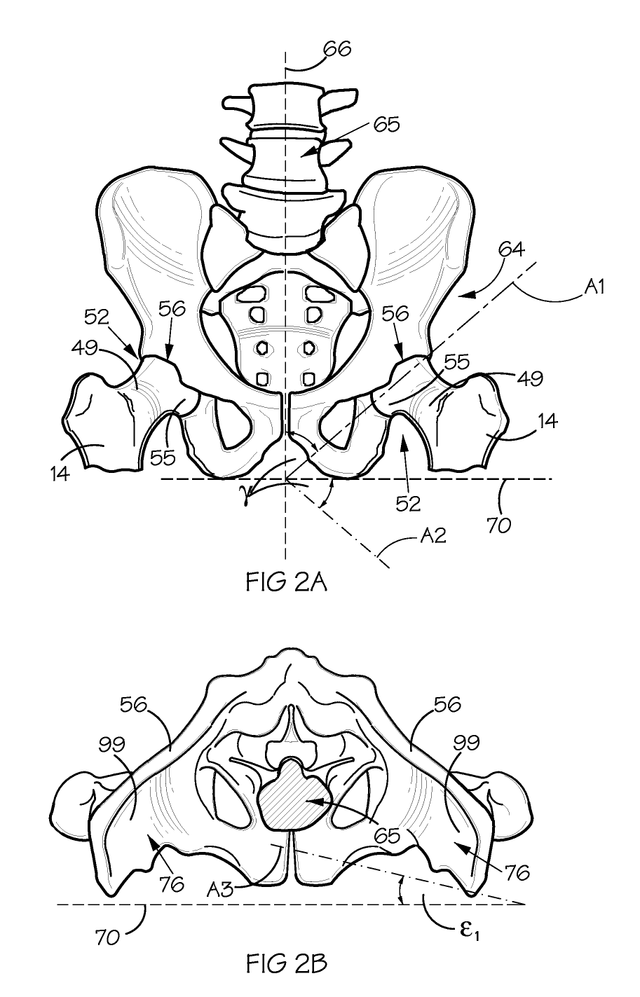

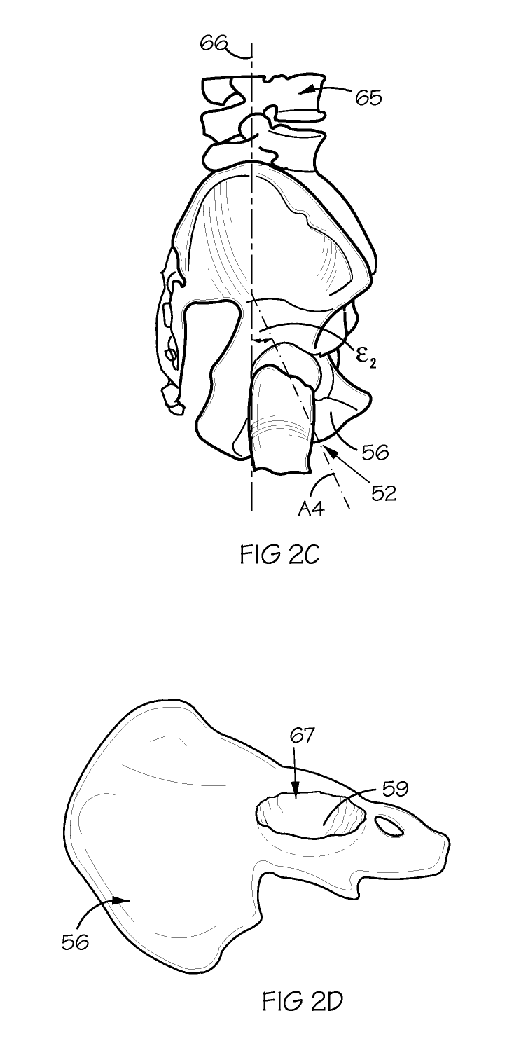

FIGS. 2A and 2C show anatomically normal hip joints 52 and a human pelvis 64. The drawings show hip bones 56 and the femur 14 inserted into an acetabulum 59 in its normal position for each hip joint 52. FIG. 2A shows a lower end region of the human spine 65, which defines a longitudinal axis 66 and a transverse axis 70 which extends perpendicularly to the longitudinal axis 66. An abduction angle γ is defined between the longitudinal axis 66 of the spine and an abduction axis A1 of approximately 45°. The abduction axis A1 is disposed perpendicularly with respect to a second abduction axis A2.

FIG. 2B shows a top view, of the pelvis of FIG. 2A, showing a medial wall 99 of the hip bone 56 and a first anteversion angle ε1 defined between the transverse axis 70 and an anteversion axis A3, the significance of which will be described below.

FIG. 2C shows a side view of the pelvis of FIG. 2A showing a second anteversion angle ε2 defined between the longitudinal axis 66 and an anteversion axis A4, the significance of which will be described below.

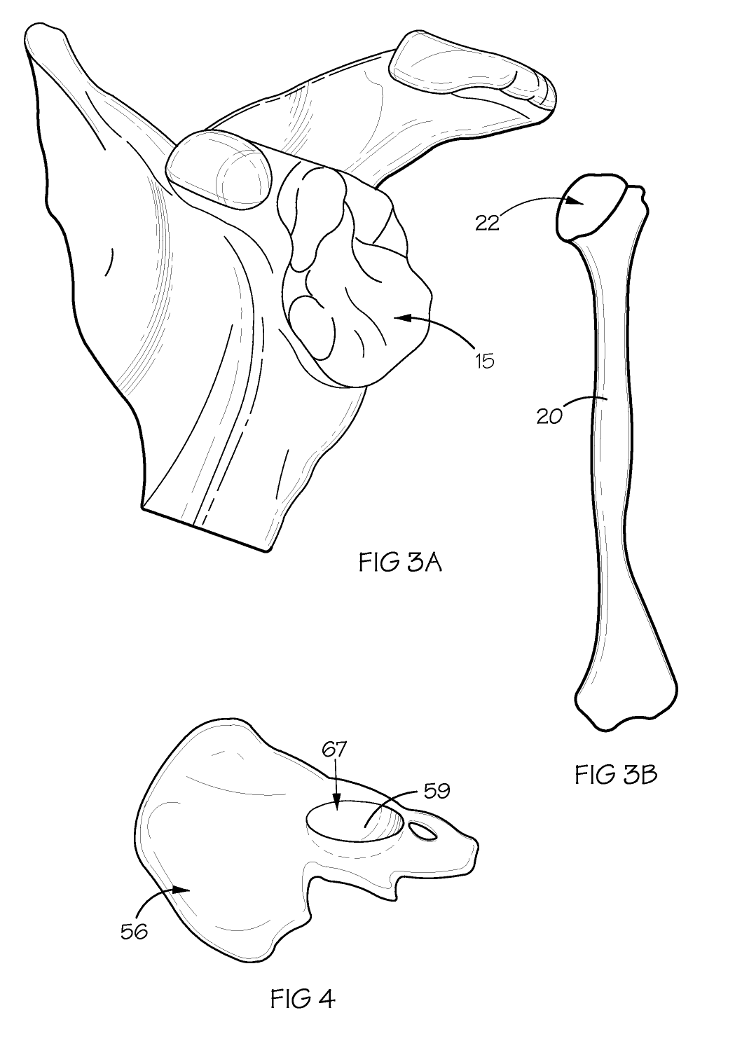

FIG. 3A shows an anatomically normal scapula having an anatomically normal glenoid bone 15. FIG. 3B shows an anatomically normal humerus.

During joint replacement surgery, at least one prosthetic joint locating face must be cut in an end region of a bone by removing a portion of bone, so as to allow for the secure fitment of a prosthetic joint to the bone in a predetermined orientation which approximates the anatomical normality of the patient's joint.

During a hip joint replacement surgical procedure, the acetabulum 59 of the hip and the head 55 of the femur 14 are cut.

During a shoulder joint replacement surgical procedure, the glenoid bone 15 and the head of the humerus are cut.

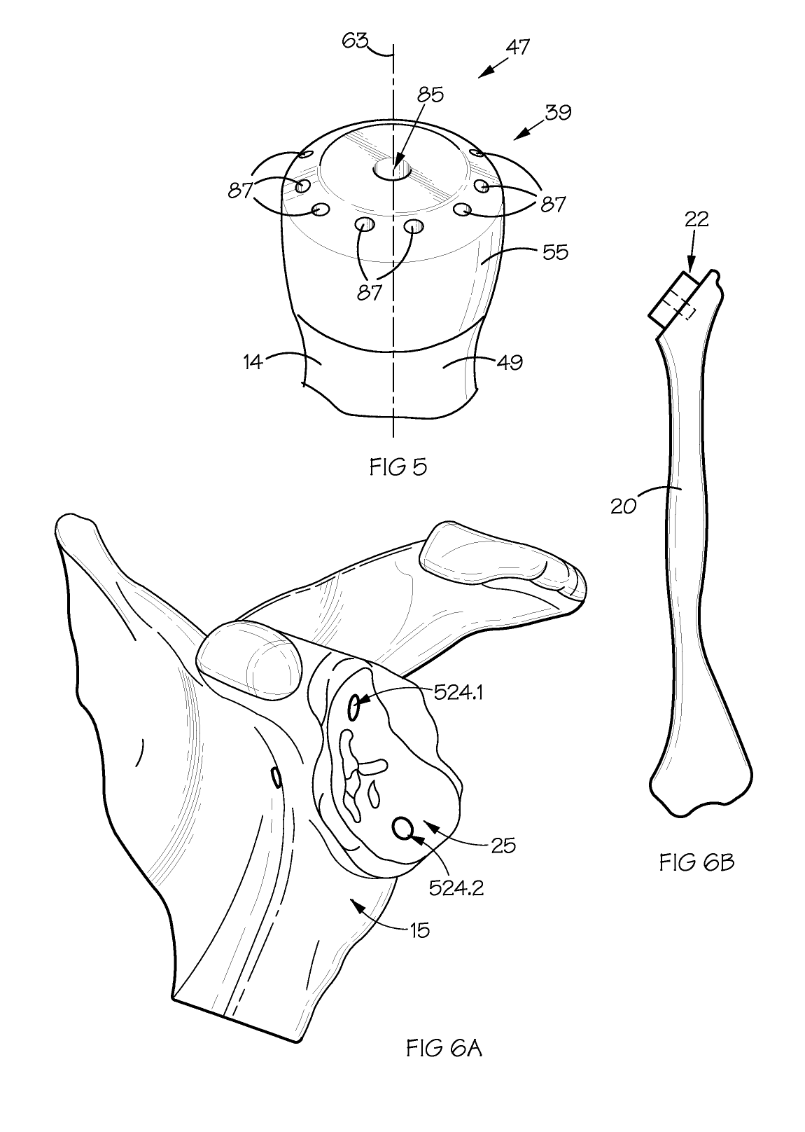

With reference to FIGS. 4 and 5, the cuts made to the acetabulum 59 and femoral head 55, respectively, in a hip joint replacement surgical procedure, are described below. FIG. 2D shows a hip bone 56 which defines an acetabulum 59 which must be cut by reaming the acetabulum to remove a portion of bone. FIG. 4 shows the acetabulum 59 which has been reamed in order to form a hemispherical cavity of uniform size to form a hemispherical joint locating face 67, the purpose of which will be described below.

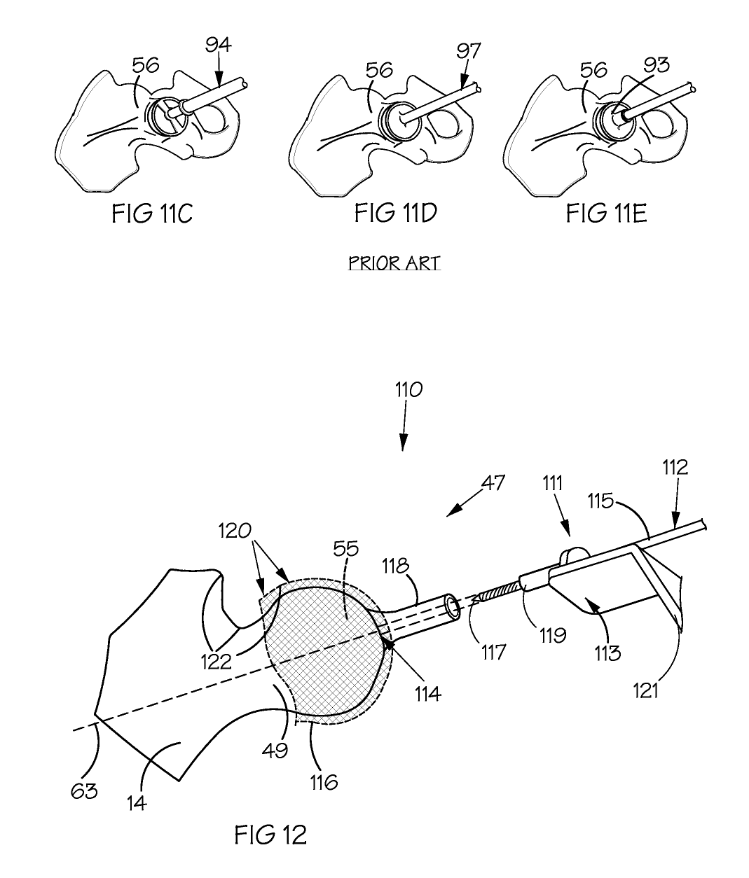

With reference to FIGS. 11A and 11B, to approximate anatomical normality, acetabular reaming must be performed in a procedure wherein the reaming tool is supported such that an axis of rotation of the reaming tool defines an angle γ of abduction of roughly 45° with the longitudinal axis 66 of the spine 65. The axis of rotation of the reaming tool must also be disposed at an angle ε2 of anteversion of approximately 15° measured from the longitudinal axis 66 of the spine 65 as shown in the drawing. With respect to the depth of the reaming, it is critical that the medial wall of the acetabulum 59 must not be penetrated.

FIG. 5 shows a cut head 55 of the femur 14 showing a prosthetic joint locating face 39 formed by a reamed end region 47 of the head 55 of the femur 14 and a hole 85 drilled into the head 55, along the implant axis 63. A number of holes 87 are shown drilled into the head 55 of the femur 14, the purpose of which will be described below.

Referring to FIGS. 6A and 6B of the drawings. FIG. 6A shows a cut glenoid bone 15 which has been cut by reaming the glenoid bone 15 to form an irregular substantially ovaloid cavity, thereby forming a joint locating face 25. A pair of holes 524.1 and 524.2 are drilled into the glenoid bone 15, the purpose of which will be described below.

FIG. 6B shows a cut head 22 of a humerus 20 showing a prosthetic joint locating face formed by reaming the head of the humerus.

The joint locating faces 39 and 67 cut into the hip joint bones as described hereinabove, allow the secure fitment of a prosthetic joint to the bones in a predetermined orientation which approximates the anatomical normality of the hip joint.

A number of different joint prostheses are supplied by different manufacturers. Different manufacturers require different cuts to be made to the relevant bone to form joint locating faces in order to fit their particular prosthesis.

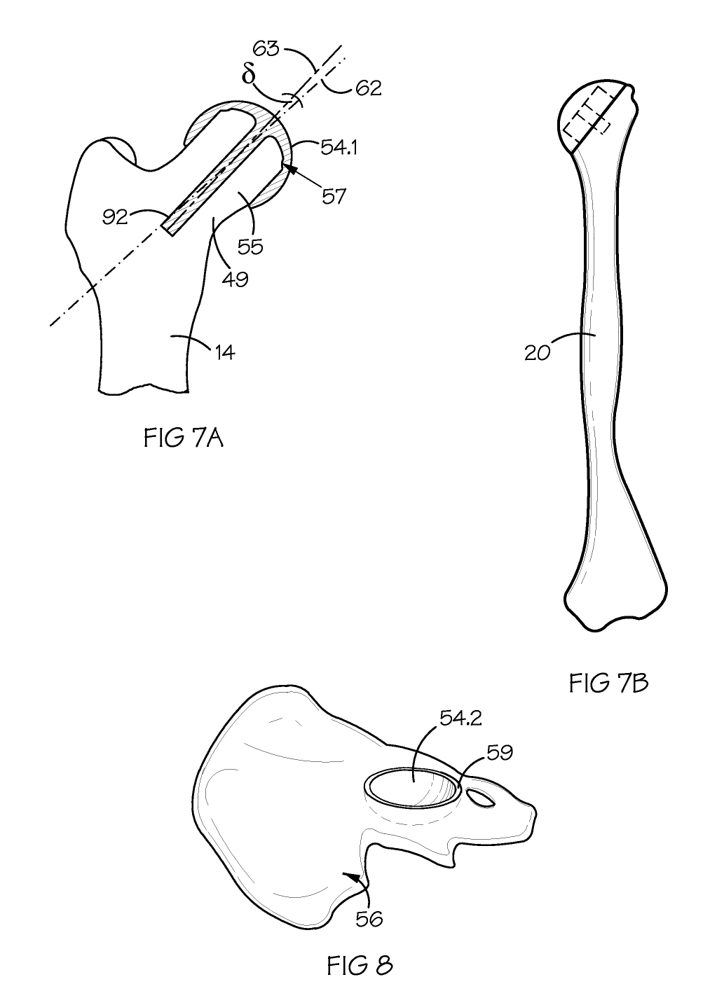

FIGS. 7A and 8 show a typical prior art hip prosthesis which includes a femoral component 54.1 and an acetabular component in the form of an acetabular cup 54.2.

FIG. 7A shows the femoral component 54.1 connected to the cut head 55 of the femur 14 in a configuration which approximates the anatomical normality of the hip joint 52. The femoral component 54.1 includes a generally cup-shaped receiving formation 57 into which the cut head 55 of the femur 14, is received, in use. The femoral component 54.1 also includes an implant pin 92 extending from the receiving formation 57, as is shown FIG. 7A. The implant pin 92 is inserted into the hole 85 which is drilled along the implant axis 63. FIG. 8 shows the acetabular cup 54.2 implanted into the prosthetic joint locating face 67 cut in the acetabulum 59. The acetabular cup 54.2 is hemi-spherical and defines a receiving formation for receiving the femoral component 54.1 therein in a configuration which approximates the anatomical normality of the patient's hip 52 joint.

FIG. 9 shows a glenoid bone 15 to which a prosthesis in the form of a glenoid implant 23 has been fitted by insertion of the implant 23 into the joint locating face 25 which was cut into the glenoid bone 15. The glenoid implant 23 is secured to the glenoid bone 15 by means of a pair of screws 25.1, 25.2 which are inserted through apertures provided therefor in the implant 23 and which are fixedly secured to the glenoid bone 15. The implant 23 includes a generally cup-shaped receiving formation into which the humerus component of the shoulder prosthesis is received.

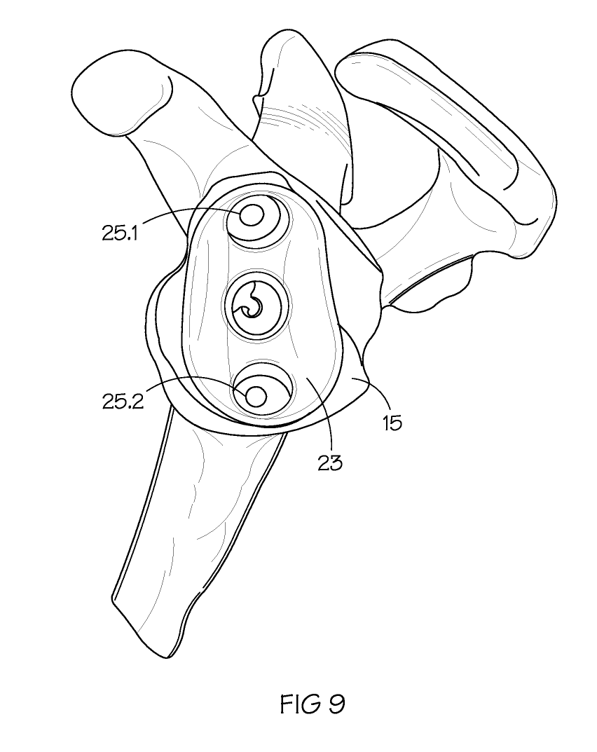

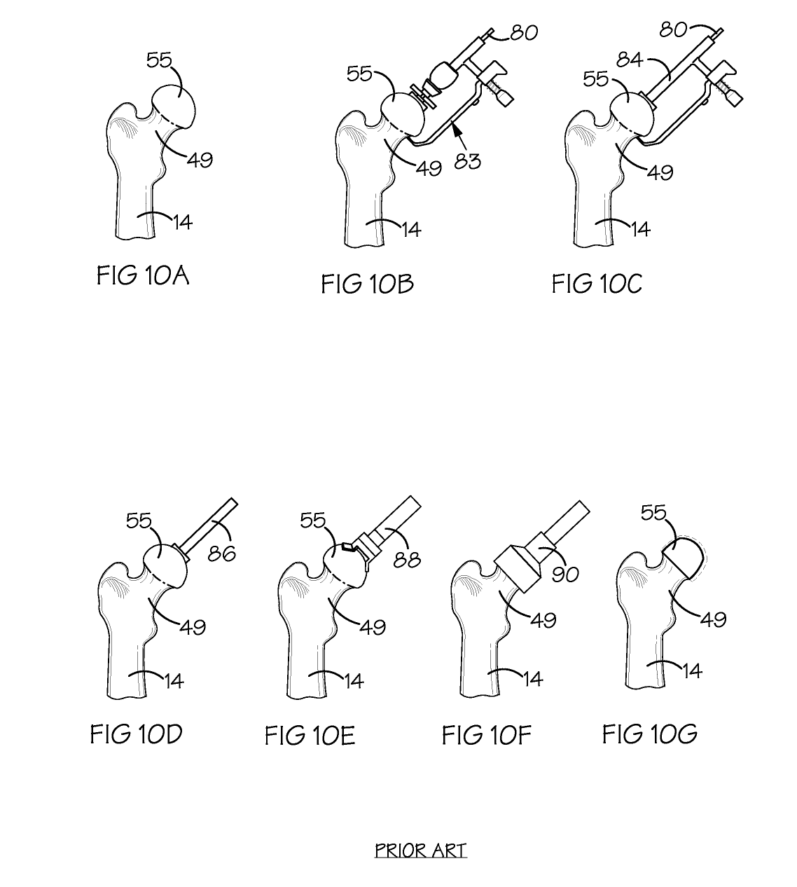

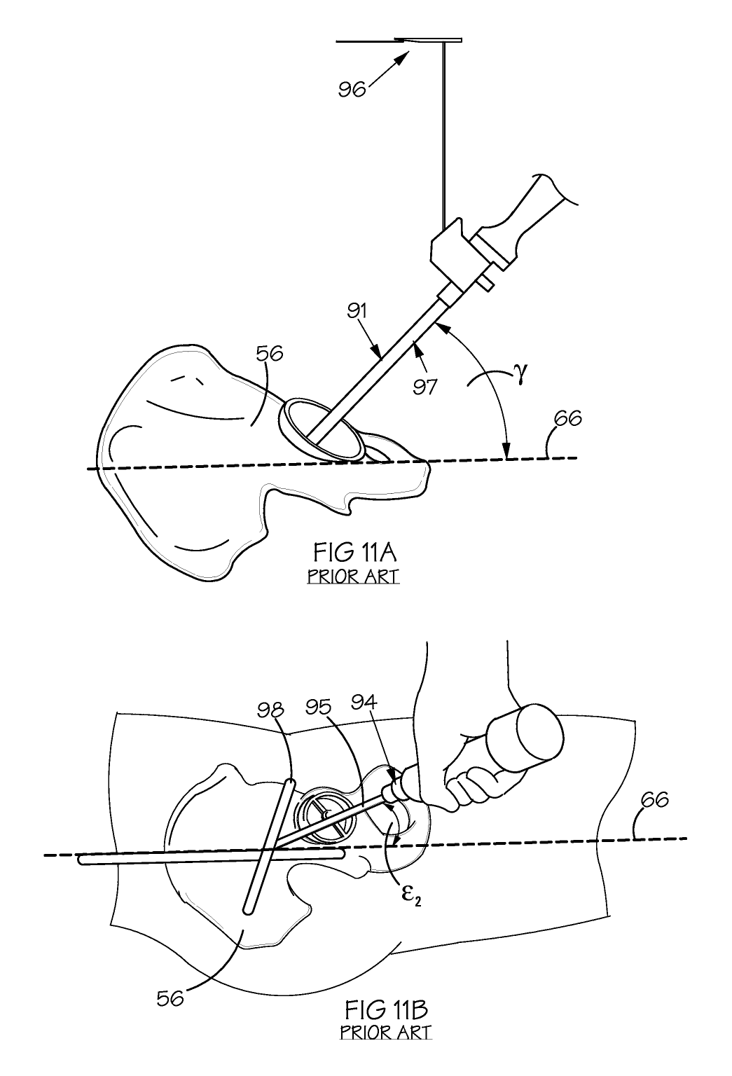

With reference to FIGS. 10 A to G, the cutting of the head 55 of the femur 14 of the hip joint is described in sequence, using a prior art hip cutting guide system 83 for cutting the head 55 of the femur 14. The prior art hip cutting guide system 83 includes a reference pin 80; a pin reamer 84 for cutting a hole for inserting a guide pin 86, which acts as a guide for guiding a chamfer reamer 88 and a profile reamer 90.

To commence the hip replacement procedure, the patient is positioned on an operating table in a lateral decubitus position, using table supports placed against the sacrum and anterior superior iliac spine (not shown) to ensure proper alignment of the patient's spine 65. Critically, the transverse axis 70 of the patient's spine 65 must be at right angles to the table and the pelvis must not be excessively flexed. The underlying leg is placed straighter than the leg to be operated upon and the knee joint and hip joint 52 of both legs being flexed at roughly 45°. The patient is anesthetised and incisions are made to the patient's hip region to expose the hip joint 52 whereafter the hip joint 52 is dislocated.

With reference to FIGS. 10A to 10I, the cutting of the head 55 of the femur 14 during a standard prior art hip replacement surgical procedure, is illustrated in sequence.

With reference to FIG. 10B, the surgery involves, as a first step, the insertion of the reference pin 80, which is inserted along the implant axis 63 (shown in FIG. 7A), which will determine the eventual alignment of the femoral implant 54.1. The alignment is estimated visually by the surgeon, with the aid of the guiding device 83, used to estimate and guide the insertion of the reference pin 80.

Referring to FIG. 10C, after insertion of the reference pin 80, the pin-reamer 84 is fitted to the reference pin 80. The pin-reamer 84 uses the reference pin 80 as a guide to guide the drilling of a reference hole into the femoral head 55. The central reference hole will determine the alignment of the femoral implant 54.1 as described more fully below.

Referring to FIG. 10D, after pin-reaming, the reamer-guide pin 86 is inserted into the hole formed by the pin-reamer 84. The guide pin 86 acts as a guide for guiding the chamfer reamer 88 as shown in FIG. 10E. The chamfer reamer 88 reams and shapes the head 55 of the femur 14.

As shown in FIG. 10F, the profile-reamer 90 is guided along the guide pin 86 to guide the cutting of the femoral head 55. FIGS. 5 and 10G show the femoral head 55 reamed in accordance with the above described procedure. Once the head 55 of the femur 14 has been reamed, a number of holes 87 are drilled into the head 55 of the femur 14 to provide for improved adhesion of the cement used to bond the femoral component 54.1 to the head 55 of the femur 14.

Referring to FIGS. 11A to 11E, the preparation of the acetabulum 59 during a standard prior art hip replacement surgical procedure, is illustrated in sequence, using the prior art acetabular hip cutting guide system. The prior art acetabular hip cutting system includes guide tools 94, guide tool 97 and a reaming tool 93 for cutting the joint locating faces 67 in the acetabulum 56.

In order to determine the optimal angle for acetabular reaming, various guide tools are used, as generally illustrated in FIGS. 11A and 11B. As shown in FIG. 11A, the guiding tool 97 is use to assist in the estimation of the abduction angle γ. The guide tool 97 includes an elongate shaft 91 and a parallel guide 96 connected to the shaft 91. The abduction angle γ is approximated by holding the parallel guide 96 parallel to the floor.

As shown in FIG. 11B, the guiding tool 94 is used to measure the angle from which reaming must be performed. Guiding tool 94 includes an elongate shaft 95 and a cross bar 98 connected at 90° to the shaft 95. The angle ε2 of anteversion is approximated by holding the crossbar 98 of the guide tool 94 parallel to the longitudinal axis 66 of the patients body as shown in FIG. 11B.

It will be appreciated that this method does not provide an accurate manner of determining the correct angle from which acetabular reaming should be optimally performed.

It will be appreciated that there is a need for an accurate means of determining the correct attachment position of the femoral component 54.1 of the prosthesis and the depth and configuration of the placement of the acetabular cup 54.2, implanted into the acetabulum 59, as well as angles ε1, ε2 and γ from which acetabular reaming should be performed.

During a shoulder replacement surgical procedure, the glenoid bone 15 and the head of the humerus must be cut. The procedures for the cutting of joint locating faces into the head of the humerus and the glenoid bone 15 will not be described in detail. It will be appreciated in this regard that the procedure for cutting the glenoid bone 15 is similar to the procedure described in relation to the acetabular reaming described hereinbefore and particularly depends to a large degree on estimation.

The prior art procedure for cutting the head of the humerus will likewise not be described in detail below. The cutting of the head of the humerus relies on estimation, to a large degree, and generally involves the reaming of the head of the humerus or the recision of the head of the humerus at an estimated angle and at an estimated position.

Having described the prior art hip and shoulder replacement prosthesis and the prior art hip replacement procedure above, various embodiments of a positioning guide and of a bone cutting guide system in accordance with the first aspect of the invention will now be described below.

With reference to FIG. 12, in a first embodiment of the invention, a bone cutting guide system in accordance with the invention, in the form of a femoral head cutting guide system, for use in hip joint replacement surgery, is designated generally by the reference numeral 110.

The bone cutting guide system 110 includes a bone cutting guide assembly 111 and a positioning guide 114.

The bone cutting guide assembly 111 includes a bone cutting guide in the form of a base pin 112 and guide mounting means comprising a pin placement guide 113.

The base pin 112 has an elongate cylindrical shaft 115 terminating in a sharp piercing point at a penetrating end 117 of the shaft 115. The penetrating end 117 of the shaft 115 has a helical screw formation defined thereon. The base pin 112 is configured to penetrate soft tissue and the helical screw formation defined on the penetrating end 117 of the shaft 115 permits the penetrating end 117 of the base pin 112 to become imbedded into a bone when the base pin 112 is rotated to screw the penetrating end 117 of the base pin 112 into the bone. The base pin 112 is thus self tapping and can be securely screwed into the bone for reasons which will become clear below.

The pin placement guide 113 has an elongate handle 121 which can be gripped by a user and terminates in a cranked end; and a hollow cylindrical guide sleeve 119 which is connected to the cranked end of the handle and which defines a guide passage extending therethrough. The guide passage defined by the hollow cylindrical guiding formation 119 is dimensioned to slidingly receive the base pin 112 therein.

The positioning guide 114 includes a bone mounting structure in the form of a moulding 116 and a cutting guide locating formation in the form of a cylindrical guide post 118 extending from to the moulding 116.

The moulding 116 is constructed from anatomical data obtained of the end region 47 of the head 55 of the femur 14 prior to surgery. The moulding 116 is thus constructed prior to the surgical procedure, from anatomical data obtained by means of a radiographic scan of the head 55 of the patient's femur 14, from which scan, a three-dimensional model of the head 55 of the patient's femur 14 is constructed. The moulding defines complementary locating formations 120 which correspond to anatomical formations 122 defined on the head 55 of the femur 14. The locating formations 120 provide for secure fitment of the moulding 116 to the end regions 47 of the head 55 of the femur 14 in a specific position. More particularly, the locating formations 120 of the moulding 116 are configured to conform to and correspond to the shape and configuration of the head 55 of the femur 14. The moulding 116 is securely fitted, in use, onto the head 55 of the femur 14 with the complementary locating formations 120 of the moulding 116 corresponding with anatomical formations 122 defined on the end region 47 of the head 55 of the femur 14.

The cylindrical guide post 118 extends from the moulding 116 and defines an aperture extending therethrough and surrounds an aperture through the moulding. The cylindrical shaft 118 is configured and dimensioned to slidingly receive the guide sleeve 119 of the pin placement guide 113 therein.

In use, the moulding 116 is fitted onto the head 55 of the femur 14. It will be appreciated that there is only one possible fitment position in which the complementary locating formations 120 defined on the moulding 116 locate against corresponding anatomical formations 122 defined on the end region 47 of the head 55 of the femur 14.

Once the moulding 116 is connected to the head 55 of the femur 14 the guiding formation 119 of the pin placement guide 113 is received in the aperture defined by the cylindrical guide post 118 and the penetrating end 117 of the base pin 112 is inserted into and received in and imbedded into the head 55 of the femur 14 and secured thereto.

The guide post 118 thus provides for the guiding of the location of the base pin 112 in a predetermined position and spatial orientation relative to the head 55 of the femur 14. After the base pin 112 is secured to the head 55 of the femur 14, the pin placement guide 113 is removed by withdrawing the guide 113 from the guide post 118 and by sliding the pin placement guide 113 over the free end of the base pin 112. The positioning guide 114 is then removed from the head 55 of the femur 14 by withdrawing the positioning guide 114 from the head 55 of the femur 14 and by sliding the positioning guide 114 over the free end of the base pin 112.

The base pin 112 then serves as a guide pin for guiding the chamfer reamer 88 and the profile reamer 90 of the prior art hip cutting guide system 83, thereby guiding the cutting of the prosthetic joint locating faces 39 in the end region 47 of the head 55 of the femur 14 to provide for the fitment of the prosthetic joint 54.1 thereto.

Although the guide system 110 has been described in relation to the cutting of the head 55 of the femur 14 during a hip bone replacement surgical procedure, the guide system 110 may also be used in the same manner for cutting the head of the humerus bone, during a shoulder replacement surgical procedure. In this regard, it will be appreciated that the positioning guide 114 will not be used, but rather will be substituted by a positioning guide (not shown) having complementary locating formations which correspond with the head of the humerus.

The invention extends to the positioning guide 114 as described hereinabove.

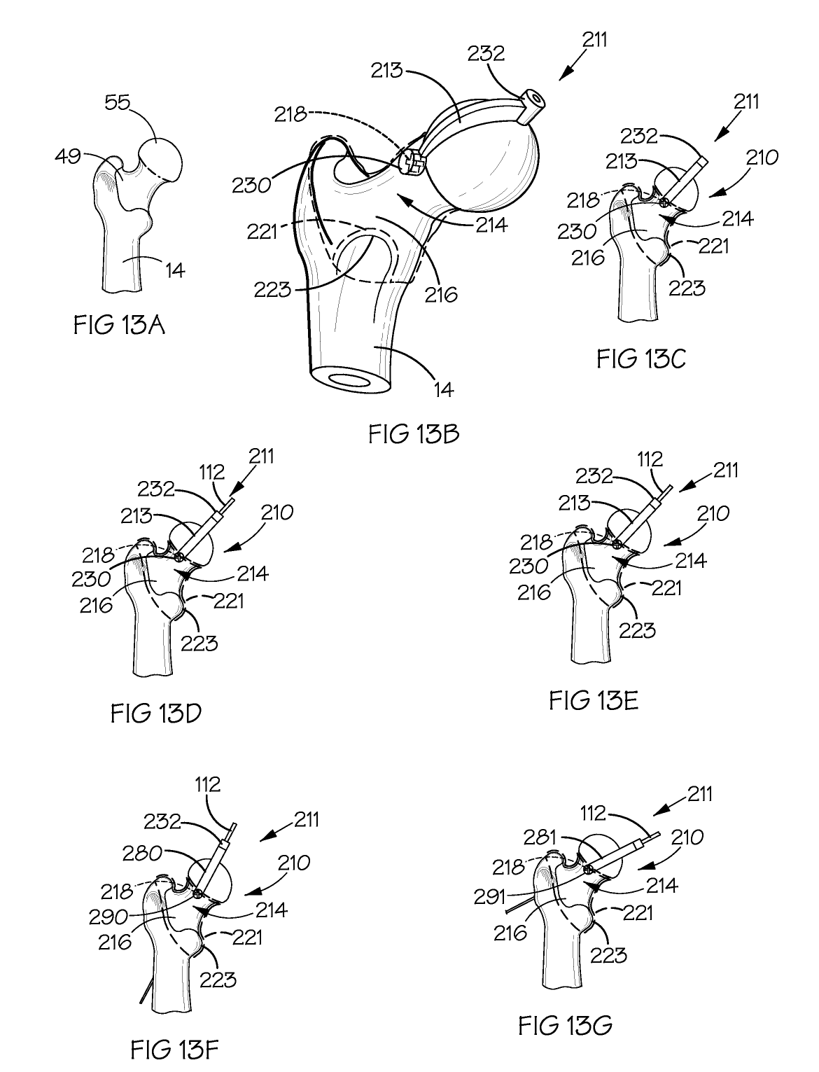

With reference to FIGS. 13A to 13E, in a second embodiment of the invention, a bone cutting guide system in accordance with the invention in the form of a femoral head cutting guide system, for use in a hip joint replacement surgical procedure, is designated generally by the reference numeral 210.

The guide system 210 includes a bone cutting guide assembly 211 and a positioning guide 214.

The bone cutting guide assembly 211 includes the base pin 112 and guide mounting means in the form of a pin placement mounting arm 213.

The mounting arm 213 has a generally C-shaped curved configuration having a complementary attachment post mounting formation 230 at one end and a pin guiding formation 232 at its opposite end.

The positioning guide 214 includes a bone mounting structure in the form of a moulding 216 and a cutting guide locating formation in the form of an attachment post 218 projecting from an external side of the moulding 216 and to which the attachment post mounting formation 230 of the mounting arm 213 can be releasably mounted as will be described in more detail below. The attachment post is cross-shaped when viewed in end view so as to provide for positive fitment of the mounting arm 213 thereto. As such, the mounting arm has a complementary cross-shaped socket formation into which the post 218 is fitted.

The moulding 216 is constructed from anatomical data obtained of the neck 49 of the femur 14 prior to surgery. The moulding 216 is thus constructed prior to the surgical procedure, from anatomical data obtained by means of a radiographic scan of the neck 49 of the patient's femur 14, from which scan, a three-dimensional model of the neck 49 of the patient's femur 14 is constructed. The moulding defines complementary locating formations 221 which correspond to anatomical formations 223 defined on the neck 49 of the femur 14. The locating formations 221 provide for secure fitment of the moulding 216 to the neck 49 of the femur 14 in a specific position. More particularly, the locating formations 221 of the moulding 216 are configured to conform and correspond to the shape and configuration of the neck 49 of the femur. The moulding 216 has a generally C-shaped configuration when viewed in end view with a degree of resilience allowing it to be securely fitted, in use, onto the neck 49 of the femur 14 with the complementary locating formations 221 of the moulding 216 corresponding with anatomical formations 223 defined on the neck 49 of the femur 14.

The pin guiding formation 232 defines a cylindrical sleeve in which the base pin 112 is slidably received.

The cross-shaped socket of the attachment post mounting formation 320, is configured to provide for the releasable mounting of the attachment post mounting formation 230 of the mounting arm 213 to the attachment post 218 of the positioning guide 214 in a predetermined orientation, thereby to orientate the mounting arm 213 relative to the attachment post 218 and thereby relative to the head 55 of the femur 14 in a predetermined position.

In use, the moulding 216 is fitted to the neck 49 of the femur 14 by alignment of the complementary locating formations 221 with anatomical formations 223 of the neck 49 in a manner similar to that described above in relation to the moulding 116. It will be appreciated that there is only one fitment position at which the complementary locating formations 221 of the moulding 216 correspond with the anatomical formations 223 defined on the neck 49 of the femur 14.

Once the moulding 216 is connected to the neck 49 of the femur 14, the mounting arm 213 is releasably mounted to the attachment post 218 of the moulding 216 via attachment post mounting formation 230 of the mounting arm 213.

It will be appreciated that there is only one possible fitment position at which the attachment post mounting formation 230 can be releasably mounted to the attachment post 218 and therefore only one possible position at which the guide mounting formation 232 is supported relative to the head 55 of the femur 14 when the mounting arm 213 is mounted to the attachment post 218 of the moulding 216 of the positioning guide 214, with the moulding 216 correctly fitted to the neck 49 of the femur 14.

Once the attachment post mounting formation 230 of the mounting arm 213 is mounted to the attachment post 218 of the moulding 216 the penetrating end 117 of the base pin 112 is inserted through the aperture defined by the pin guiding formation 232 of the mounting arm 213, which guides the insertion of the penetrating end 117 of the base pin 112 into the head 55 of the femur 14 at the predetermined position.

It will be appreciated that the configuration and the positioning of the attachment post 218 and the configuration of the mounting arm 213 are determined pre-operatively. Prior to surgery, the exact position and configuration of the attachment post 218 and the configuration of the mounting arm 213 are predetermined, thereby to determine the exact position and orientation of the base pin 112 which is inserted, in use, into the head 55 of the femur 14 when the moulding 216 is correctly fitted to the neck 49 of the femur 14. As is the case with the guide system 110, the base pin 112 of the guide system 210 which is inserted into the head 55 of the femur 14 may also serve as a guide pin. More particularly, the base pin 112 may serve as a guide pin for guiding the chamfer reamer 88 and the profile reamer 90 of the prior art hip cutting guide system 83, thereby guiding the cutting of the prosthetic joint locating faces 39 in the end region 47 of the head 55 of the femur 14 to provide for the fitment of the prosthetic joint 54.1 thereto.

The bone cutting guide assembly 211 of the guide system 210 may also include a selection of differently configured mounting arms 280, 281, each mounting arm 280, 281 being similar to mounting arm 213, but the configuration of the attachment post mounting formations 290, 291 of attachment arms 280, 281, respectively, differs. More particularly, each different mounting arm 213, 280, 281 provides for a different position at which the guide mounting formation of said arm is supported relative to the head 55 of the femur 14 when said attachment post mounting formation of said mounting arm is connected to the guide shaft 118 and thereby to the head 55 of the femur 14. In use, a different one of the selection of guide arms 213, 280, 281 may be selected to be connected to the moulding 216, for adjusting the placement of the guide mounting formations of said guide arm relative to the head 55 of the femur 14 and thereby adjusting the exact position and orientation of the base pin 112 which is inserted into the head 55 of the femur 14.

With reference to FIGS. 13E, 13F and 13G, it will be appreciated that the selection of a different one of the mounting arms 213, 280, 281 permits a surgeon to adjust the placement of the base pin 112 during a surgical procedure by selecting an appropriate one of the mounting arms which will provide for a particular degree of adjustment, to allow the surgeon to change the position of insertion and angle of insertion of the base pin 112 intra-operatively.

Although the guide system 210 has been described in relation to the cutting of the head 55 of the femur, during a hip joint replacement surgery, the guide system 210 can be used in the same manner for cutting the head of the humerus during shoulder replacement surgery, with the only difference being that the positioning guide 214 is substituted with a positioning guide (not shown) configured for fitment to the neck of the humerus.

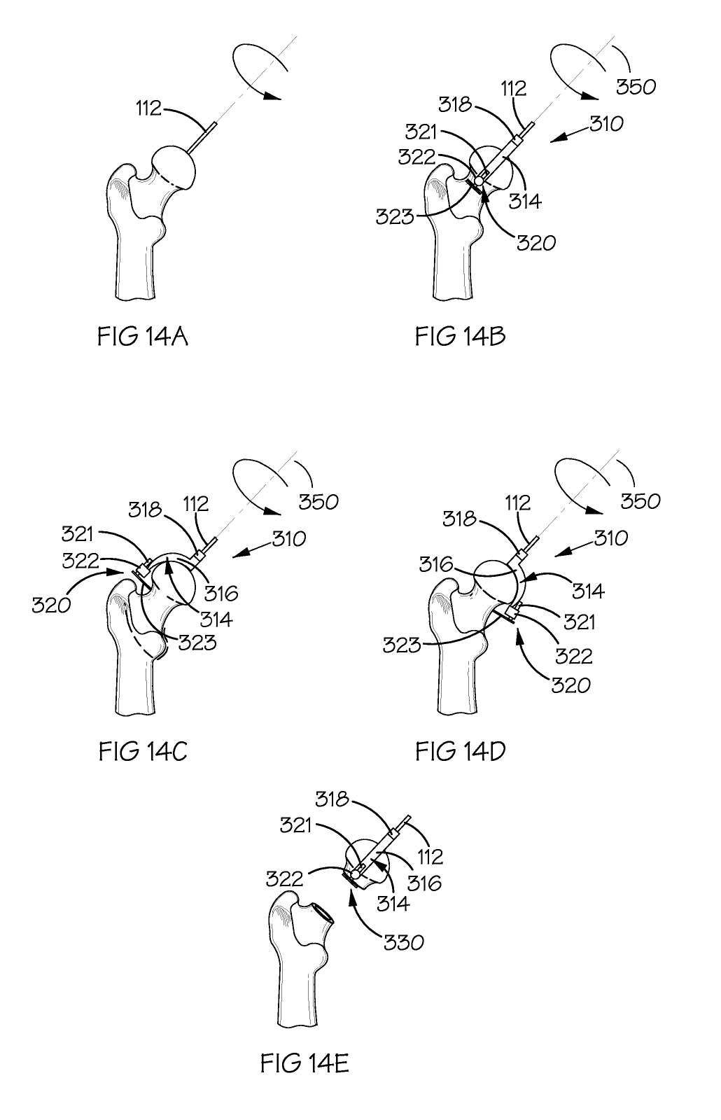

With reference to FIGS. 14A to 14E in a third embodiment of the invention, a bone cutting guide system in accordance with the invention in the form of a femoral bone cutting guide system, for use in a femoral head replacement surgical procedure is designated generally by the reference numeral 310.

The bone cutting guide system 310 includes the bone cutting guide system 210 and a rotatable cutter guide 314.

The rotatable cutter guide 314 comprises a body 316 and a cutter assembly 320 mounted to the body 316.

The body 316 has a plate-like configuration and defines a pin mounting formation 318 at a first end of the body and a cutter mounting formation 322 at a second end of the body 316. The pin mounting formation 318 defines a cylindrical sleeve in which the base pin 112 is slidingly received, for rotatably mounting the cutter assembly 320 to the base pin 112.

The cutter assembly 320 comprises a mounting arm 321 connected to a cutter guide formation 323. The mounting arm 321 is adjustably received in the cutter assembly mounting formation 322 of the cutter guide 314.

With reference to FIGS. 14A to 14E, the use of guide system 310 will be described below in a step-by-step fashion.

In use, the procedure for inserting the base pin 112 into the head 55 of the femur 14 by using the positioning guide 214 and the pin placement arm 213, is the same procedure as that described in relation to the femoral head bone cutting guide system 210. FIG. 14A shows the head 55 of the femur 14 to which the base pin 112 has been inserted as described above in relation to system 210.

Once the base pin 112 is inserted into the head 55 of the femur 14, the procedure for re-sectioning of the femoral head 55 can commence. To commence the re-sectioning procedure, with reference to FIG. 14B, the cutter guide 314 is rotatably mounted to the base pin 112 by receiving the base pin 112 through the aperture defined through the base pin mounting formation 318.

The position of the cutter guide formation 323 relative to the head 55 of the femur 14 is adjusted by adjusting the position at which the mounting arm 321 is fixedly secured to the cutter assembly mounting formation 322 to an appropriate position to guide the cutting of the neck 49 of the femur 14, at a desired position.

As shown in FIGS. 14B to 14D, the cutter guide 314 is rotatable about an axis 350 of rotation which coincides with a longitudinal axis of the base pin 112. The cutter guide 314 is rotated about its swivel attachment to the base pin 112 by means of its base pin mounting formation 318 such that the cutting formation 323 revolves around the neck 49 of the femur 55. A cutter (not shown) is guided by the cutting formation 323 to guide the cutting of the neck 49 of the femur 14. The cutting procedure continues until the neck 49 of the femur 14 is cut through, as illustrated in FIG. 14E.

In use, the cutting guide system 310 guides the cutting through of the neck of the femur 14. It will be appreciated that similar to the cutting guide systems 110 and 210, the cutting guide system 310 can likewise be used for cutting through the neck of the humerus. Although the guide system 310 has been described in relation to the cutting of the head of the femur 14, during a hip joint replacement surgery, the guide system 310 can also be used for cutting the head of the humerus during a shoulder replacement surgery. In this regard, it will be understood that the positioning guide 214 will not be used, but rather will be substituted by a positioning guide (not shown) having complementary locating formations which correspond with the neck of the humerus.

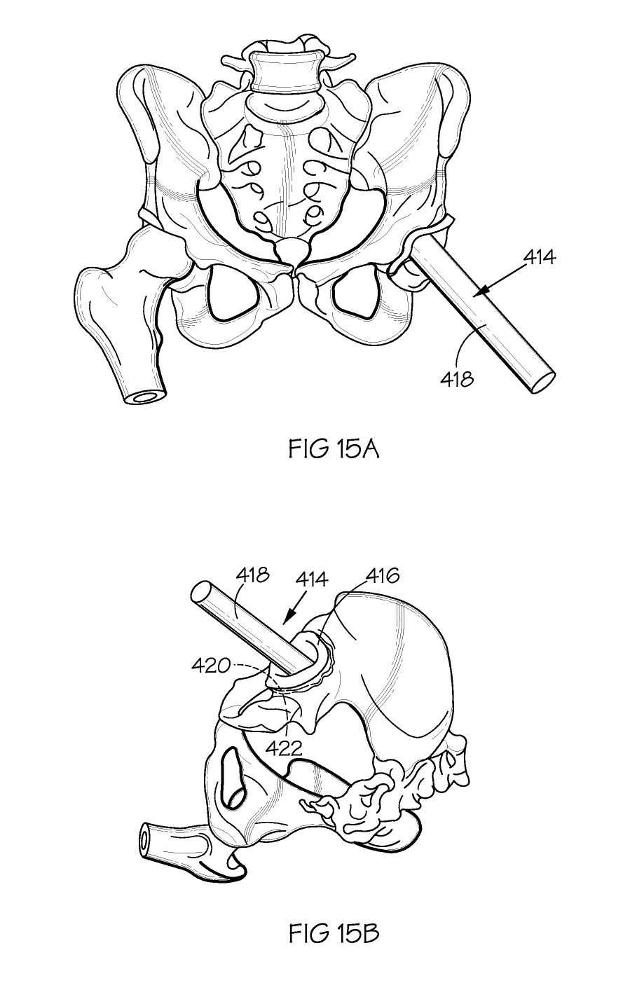

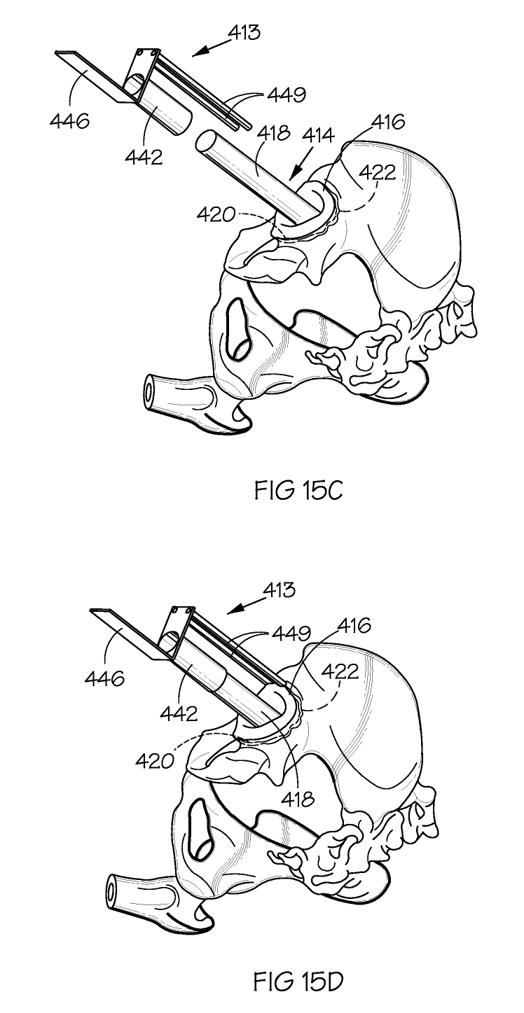

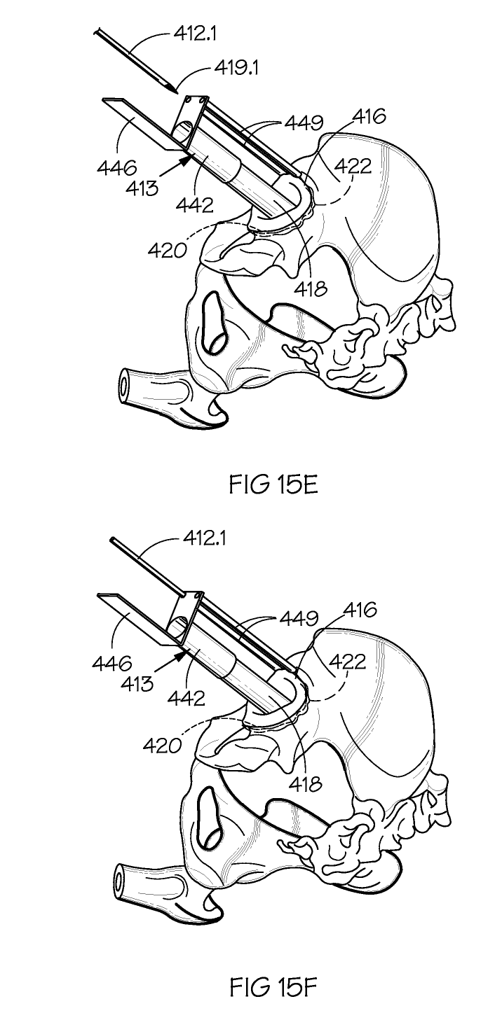

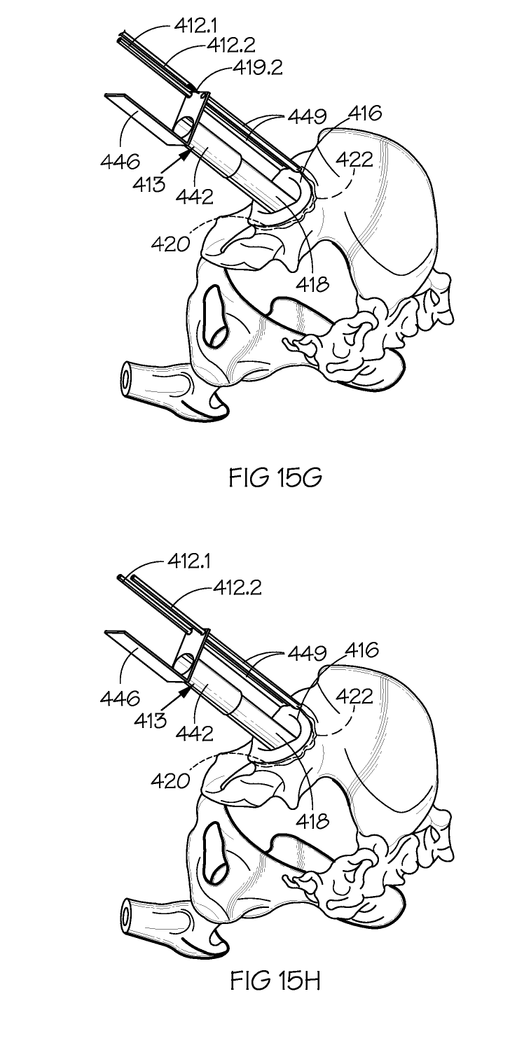

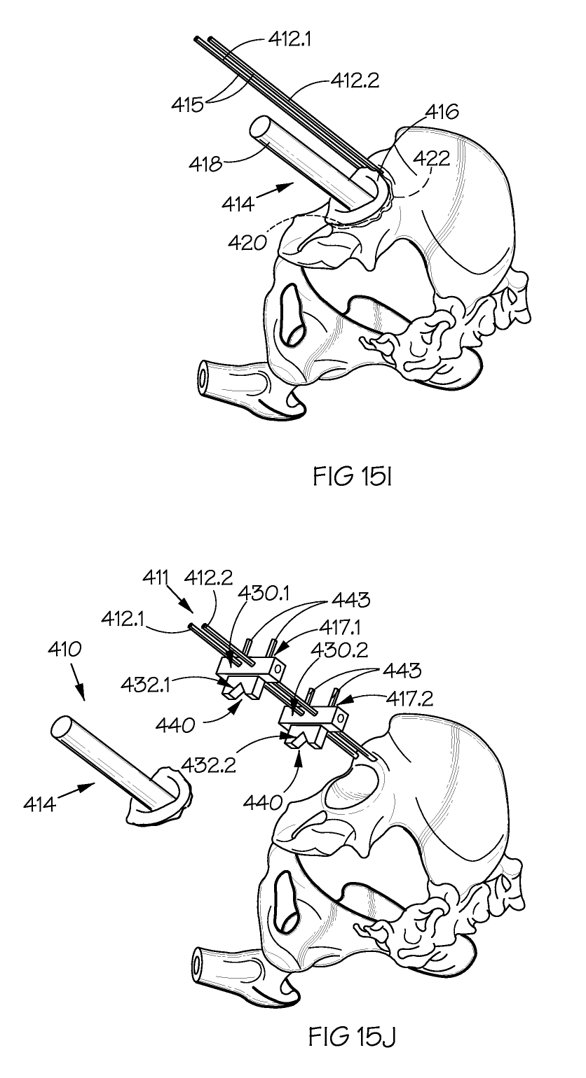

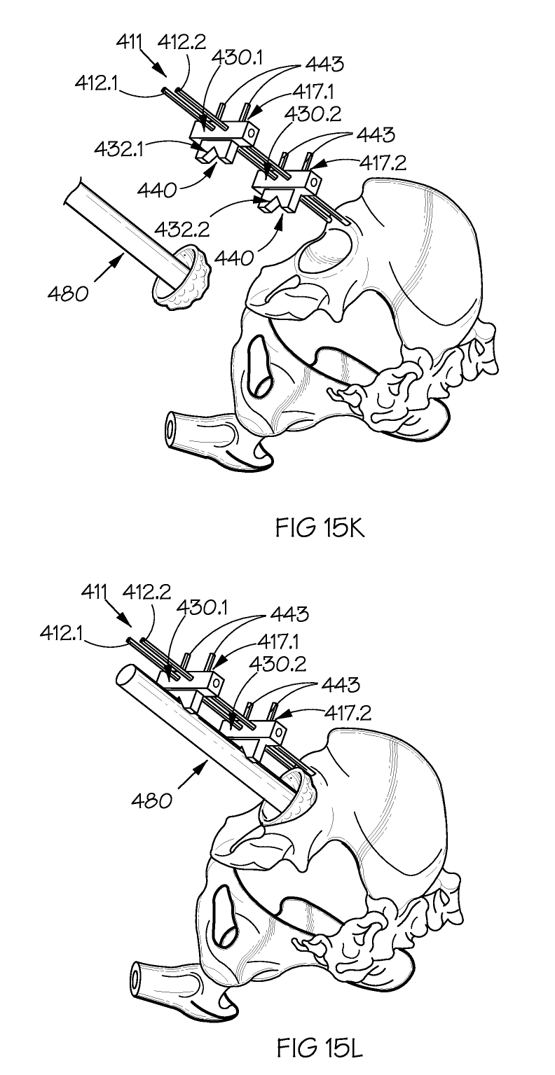

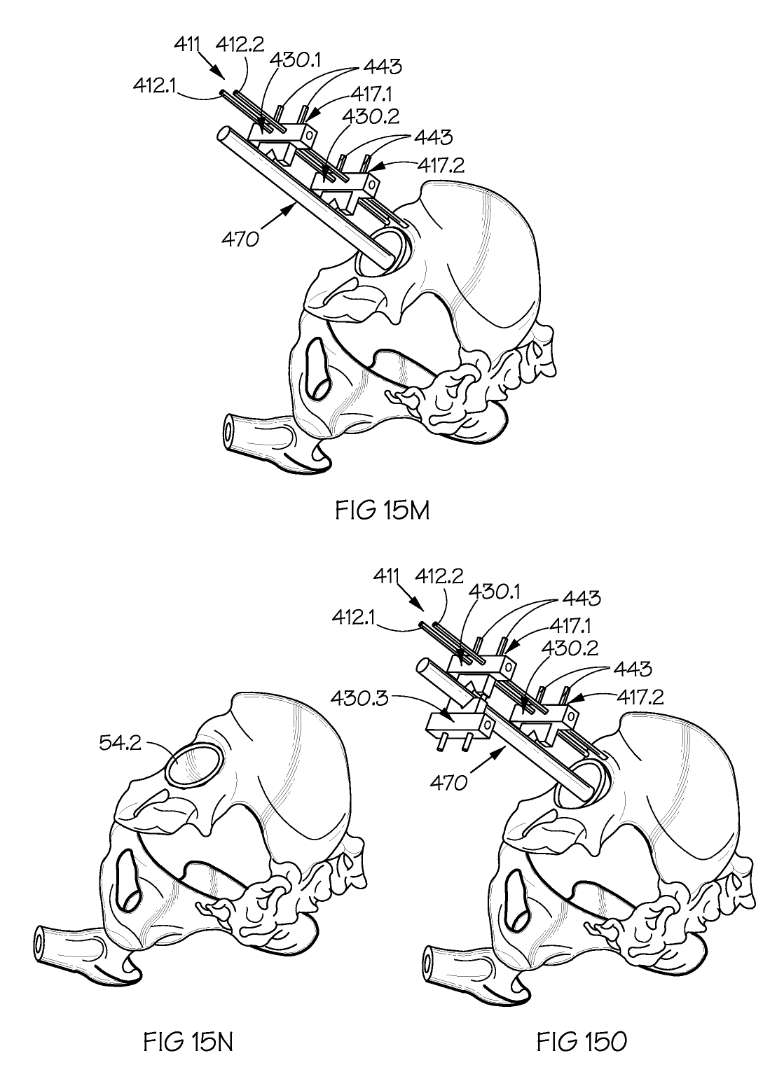

With reference generally to FIGS. 15A to 15J and with specific reference to FIG. 15J, in a fourth embodiment of the invention, a bone cutting guide system in accordance with the invention, in the form of an acetabular bone cutting guide system, for use in hip joint replacement surgery, is designated generally by the reference numeral 410.

The guide system 410 includes a bone cutting guide assembly 411 and a positioning guide 414.

The bone cutting guide assembly 411 includes a pair of guide block assemblies 417.1, 417.2 and a pair of guide pins 412.1, 412.2 and guide mounting means comprising a pin placement guide 414.

Each guide block assembly 417 includes a pair of base blocks 430.1, 430.2 and a pair of V-blocks 432.1, 432.2.

Each base block formation 430.1, 430.2 comprises a metal rectangular body.

Each of the V-blocks 432.1, 432.2 are of metal and comprise a V-shaped guide formation 440 and a pair of mounting posts 443 extending from one side of the guide formation 440. The posts 443 are adjustably received in holes defined in the blocks 430 for adjustably mounting the V-shaped guide formations 440 to the base blocks 430 as shown in FIG. 15J of the drawings.

The guide pins 412 are slidingly received in holes defined therefor in the base blocks so as to allow the blocks 430 to slide along the length of the guide pins as shown in FIG. 15J.

Each guide pin 412 has an elongate shaft and a piercing point 419 at a penetrating end 419 of the pin. The penetrating end 419 of the pin 412 has a self-tapping screw thread.

Each pin 412 is configured to penetrate soft tissue and the screw thread permits the penetrating end 419 to be screwed into the bone.

The pin placement guide 413 comprises an attachment post mounting formation in the form of a cylindrical sleeve 442, a pair of spaced apart cylindrical guide tubes 449 and an L-shaped plate 446 connected between upper ends of the sleeve 442 and the guide tube 449. The cylindrical guide tubes 449 are each dimensioned to slidingly receive a different one of the pins 412 therein.

Referring to FIG. 15I, the positioning guide 414 includes a bone mounting structure in the form of a moulding 416 and a cutting guide locating formation in the form of an attachment post 418 extending from an external side of the moulding 416.

The moulding 416 is constructed from anatomical data obtained of the patient prior to surgery. The moulding 416 is thus constructed prior to the surgical procedure, from an anatomical data obtained by means of a radiographic scan of the acetabulum of the patient's pelvis, from which scan, a three dimensional model of the acetabulum 59 of patient's pelvis is constructed. The moulding defines complementary locating formations 420 which correspond to anatomical formations 422 defined on the acetabulum. The locating formations 420 provide for secure fitment of the moulding 416 to the acetabulum 59 in a specific position. More particularly, locating formations 420 of the moulding 416 are configured to conform to and correspond to the shape and configuration of the acetabulum 59. The moulding 416 is securely fitted, in use, to the acetabulum 59 with the complementary locating formations 420 of the moulding 416 corresponding with anatomical formations 422 defined on the acetabulum 59.

The attachment post 418 is configured and dimensioned to be slidingly received in the cylindrical sleeve 442 of the pin placement guide 413.

In use, the moulding 416 is fitted into the acetabulum 59. It will be appreciated that there is only one possible fitment position in which the complementary locating formations 420 defined on the moulding 416 locate against corresponding anatomical formations 422 defined by the acetabulum 59. Once the moulding 416 is connected to the acetabulum 59 the sleeve 442 of the pin placement guide 413 is received on the attachment post 418 as shown in FIGS. 15C to 15D. The pin placement guide 413 is rotatably mounted to the attachment post 418, thereby permitting the surgeon to rotate the guide 413 until a suitable attachment position is found at which the pins 412 are inserted into the bone. With reference to FIGS. 15E to 15G, the cylindrical sleeve 442 is rotatably located on the attachment post 418. This provides for the rotation of the guide 413. The guide pins 412.1 and 412.2 are then inserted into the cylindrical guide tubes 449 of the pin placement guide 413. The guide pins 412.1 and 412.2 are each screwed into the hip bone at a suitable location which is found by rotating the guide 413, which is selected to minimise damage to the patient's soft tissue, which provides secure attachment to hard bony areas.

The cylindrical tubes 449 of the pin placement guide 413 therefore provide for the guiding of the locating of the guide pins 412.1 and 412.2 in a predetermined position and spatial orientation relative to the acetabulum 59, yet allowing the surgeon to select the most appropriate attachment point.

With reference to FIGS. 15H and 15I, after the guide pins 412.1 and 412.2 are secured to the acetabulum 59, the pin placement guide 413 is removed by withdrawing the pin placement guide 413 from the attachment post 418 by sliding the pin placement guide 413 over the free end of the attachment post 418 and over the free ends of the guide pins 412.1, 412.2.

The positioning guide 414 is then removed from the acetabulum 59 by withdrawing the positioning guide 414 from the acetabulum 59 and by sliding the positioning guide 414 over the free ends of the guide pins 412.1 and 412.2.

The guide pins 412.1 and 412.2 thus serve as guides upon which the base blocks 430.1, 430.2 can be releasably mounted by sliding the base blocks onto the guide pins via the holes defined in the base blocks therefore. The mounting post 443 of each of the V-blocks 432.1, 432.2 are inserted into the apertures defined in the base blocks therefore as shown in FIG. 15J of the drawings. The mounting posts 443 are held in position by means of screws (not shown) inserted through the apertures 438 of the base blocks.

With reference to FIGS. 15K and 15L, in use, a reaming tool 480 is guided by the V-blocks 432.1, 432.2 as shown in FIGS. 15K and 15L of the drawings, during reaming of the acetabulum 59, as shown in the drawings.

Once the reamer has cut the joint locating face in the acetabulum, the acetabular cup 54.2 is located in the reamed acetabulum 59, as shown in FIG. 15M of the drawings. Thereafter, the reamed acetabular cup is compacted by means of compacting tool 470 which is guided by the V-blocks 432.1, 432.2 for guiding the impacting of the acetabular cup 54.2 of the prosthesis into the reamed cavity formed in the acetabulum 59.

It will be appreciated that the V-blocks 432 of the guide block assemblies 417 are adjustably mounted to the guide pins 412 to permit sliding along the guide pins 412, thereby to permit the cutting block assemblies 417 and guide pins 412 to guide reaming tools of different configurations and sizes. It will also be appreciated that the cutting block assemblies 417 and guide pins 412 permit the reaming tool 480 to “kick-out”, i.e. to move away from the V-shaped guide formations 440, if the reaming blade of the reaming tool 480 “snags” on a hard region of bone. This arrangement permits the reaming tool 480 to be deflected away from the V-shaped guide formations 440 without moving the bone cutting guide 415 from its secure attachment to the bone.

In another arrangement shown in FIG. 15O, the reaming tool 480 can be clamped by the addition of an additional guide block 430.3 which is secured to guide block 430.1, as shown in FIG. 15O. This arrangement permits for more secure guiding of the reaming tool 480 during a reaming operation.

In this arrangement, guide block 432.2 acts as a guide stop which prevents further reaming when guide block 432.1 is stopped by guide block 432.2, thereby to prevent further penetration of the reaming tool 480, thereby to prevent the penetration of the medial wall 99 of the acetabulum 59.

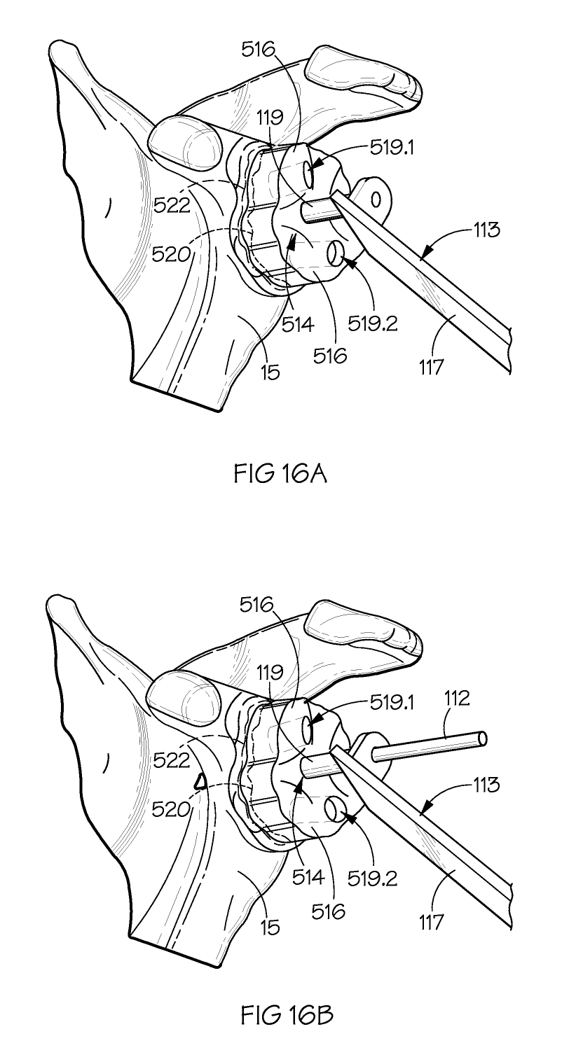

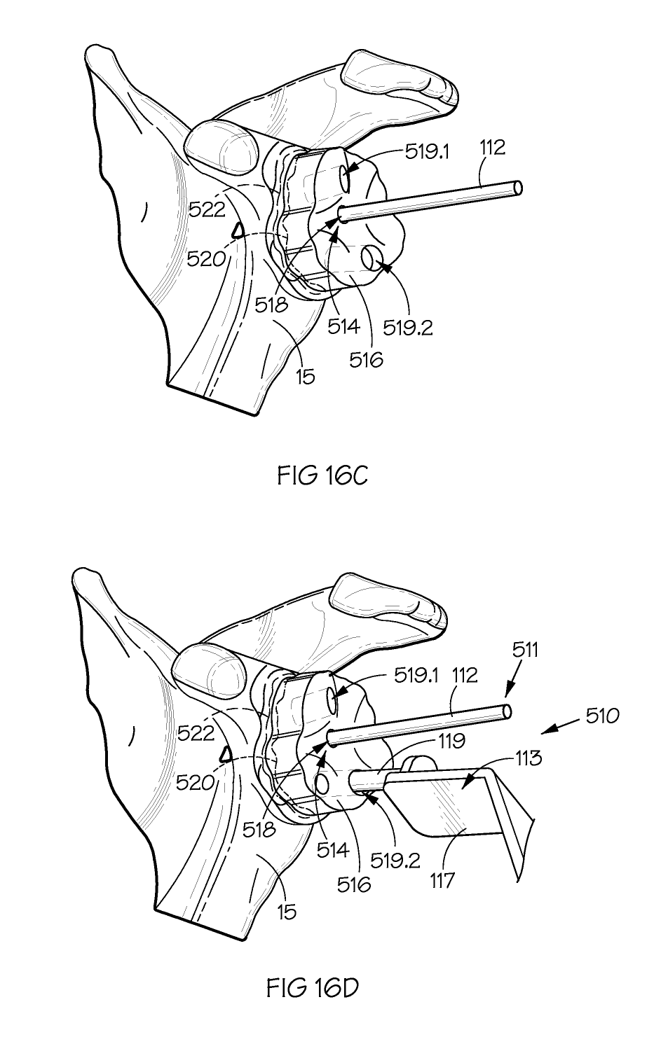

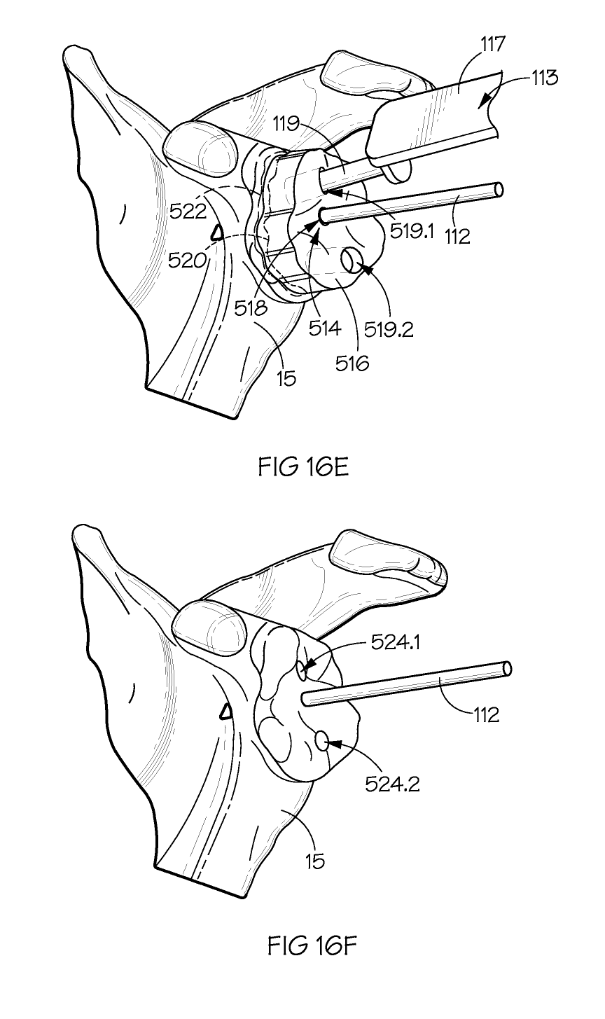

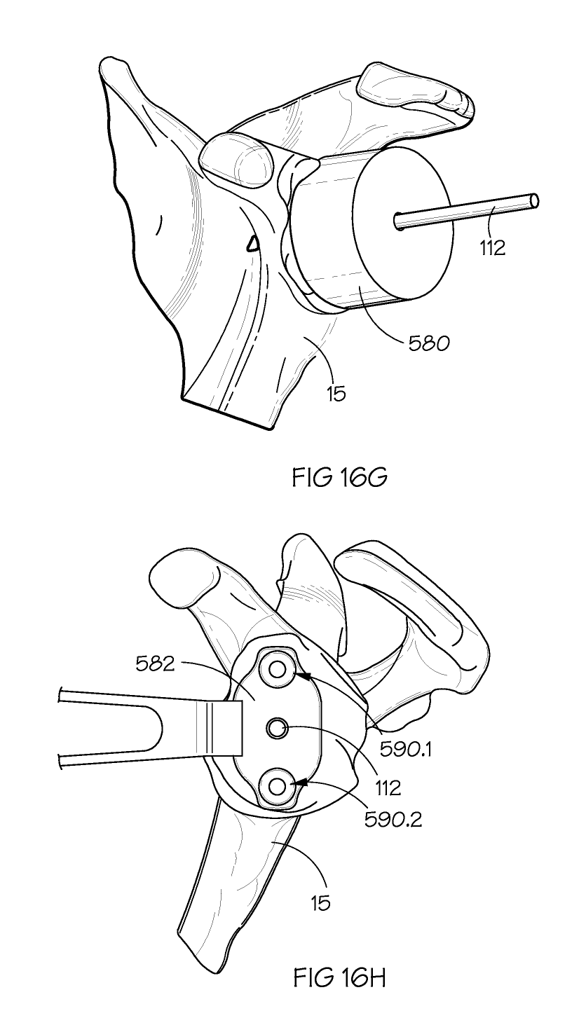

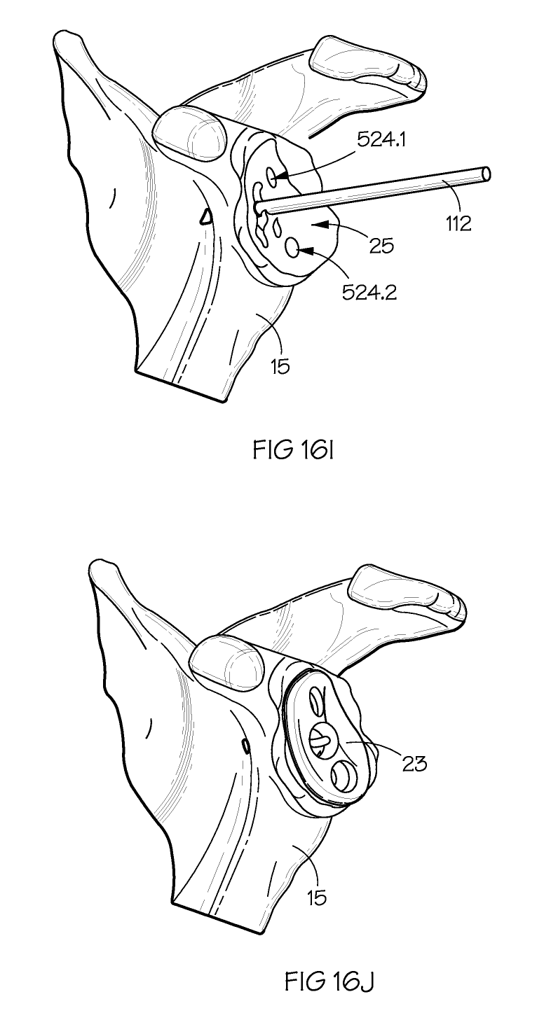

With reference to FIGS. 16A-16J, in a fifth embodiment of the invention, a bone cutting guide system in accordance with the invention, for use in a shoulder joint replacement surgery, is designated generally by the reference numeral 510. The bone cutting guide system 510 is adapted for use in cutting a prosthetic joint locating face in the glenoid bone of a patient The bone cutting guide system 510 includes a bone cutting guide assembly 511 and a positioning guide 514.

The bone cutting guide assembly 511 includes a bone cutting guide in the form of the base pin 112 and guide mounting means comprising the pin placement guide 113 of the bone cutting guide system 110.

The positioning guide 514 includes a bone mounting structure in the form of a moulding 516 and cutting guide locating formations in the form of passages 518, 519.1 and 519.2 defined by the moulding 516.

The moulding 516 is constructed from anatomical data obtained of the end region of the glenoid bone 15 prior to surgery. The moulding 516 is thus constructed prior to the surgical procedure from anatomical data obtained by means of a radiographic scan of the glenoid bone 15, of the patient's scapular, from which scan a three dimensional model of the glenoid bone 15 is constructed. The moulding 516 defines complementary locating formations 520 which correspond to anatomical formations 522 defined on the end region of the glenoid bone 15. The locating formations 520 provide for secure fitment of the moulding 516 to the end region of the glenoid bone 15 in a specific position. More particularly, the locating formations 520 of the moulding are configured to correspond to the shape and configuration of the glenoid bone 15. The moulding 516 is securely fitted, in use, onto the glenoid bone 15 with the complementary formations 520 of the moulding 516 corresponding with anatomical formations 522 defined on the end region of the glenoid bone 15.

The aperture 518 defined through the moulding 516 is configured and dimensioned to receive the guiding formation 119 of the placement guide 113. Apertures 519.1, 519.2 slope inwardly towards aperture 518 and are configured to guide a drill bit for drilling screw locating holes into the glenoid bone 15 as will be described below.

With reference to FIGS. 16A-16J, the use of the bone cutting guide system 510 is described in step by step fashion hereinbelow.

FIG. 16A shows the moulding 516 being fitted to the glenoid bone 15 and shows the cylindrical guide sleeve 119 of the pin placement guide 113 inserted through aperture 518 defined in the moulding 516. FIG. 16B shows the base pin 112 being inserted through the aperture defined by the cylindrical guiding formation 119 of the pin placement guide 113, to guide the insertion of the base pin 112 into the glenoid bone 15. FIG. 16D shows the base pin 112 after it has been screwed into the bone 15 and the pin placement guide subsequently removed.

FIG. 16D shows the pin placement guide 113 after removal from the pin 122 with the cylindrical guide sleeve 119 of the pin placement guide 113, inserted into the aperture 519.2 defined in the moulding 516, for guiding the drilling of a first screw locating hole into the glenoid bone 15.

FIG. 16E shows the pin placement guide 113 after removal of the guiding sleeve 119 of the pin placement guide 113 from aperture 519.2, inserted into the aperture 519.1 defined in the moulding 516, for guiding the drilling of a second screw locating hole into the glenoid bone 15.

FIG. 16F shows the glenoid bone 15 with the moulding 516 removed from the glenoid bone 15 showing holes 524.1 and 524.2 which were drilled into the glenoid bone 15 in order to accommodate fixing screws for securing the prosthesis to the glenoid bone 15 as is described in more detail below.

FIG. 16G shows the glenoid bone 15 being reamed by a reamer which is guided by base pin 112, for reaming a prosthetic joint locating face 25 in the glenoid bone 15.

In use, reaming is performed in stages, to ensure that not too much bone material is removed by reaming. It will be appreciated that the holes 524.1 and 524.2 are angled and are required in order to secure the prosthesis to the bone 15. It will be appreciated that as reaming progresses, the exposed upper ends of the holes 524.1 and 524.2 will be displaced towards each other, as they are sloped as stated above.

The holes 524.1 and 524.2 therefore serve as a means of determining reaming depth, by measuring the distance between the upper ends of apertures 524.1 and 524.2 as the bone is reamed.

After a first stage of reaming, with reference to FIG. 16H, gauge tool 582 is used to check on the reaming depth by gauging the distance between upper ends of apertures 524.1 and 524.2. Once the upper ends of apertures 524.1 and 524.2 are aligned with and visible through apertures 590.1 and 590.2 of the gauge tool, then the reaming procedure can be terminated. In use, this allows the surgeon to periodically check and gauge the reaming procedure to ensure that correct depth of reaming is being achieved.

FIG. 16I shows the glenoid bone 15 which has been reamed by the reamer to form joint locating face 25 to provide for the secure fitment of prosthesis 23 to the glenoid bone 15. FIG. 16J shows the glenoid bone 15 to which the prosthesis 23 has been fitted by means of screws screwed into the first and second screw locating holes drilled into the glenoid bone.

1. A positioning guide for use with a bone cutting guide assembly for use in a joint replacement surgical procedure for guiding the cutting of at least one prosthetic joint locating face in a region of a predetermined joint bone of a human patient, from which a portion of bone is to be removed, thereby to allow for the secure fitment of a prosthetic joint to said bone in a predetermined orientation which approximates the anatomical normality of said joint bone, the bone cutting guide assembly including a bone cutting guide having at least one cutter guide formation for guiding a cutter for cutting said prosthetic joint locating face in said region of said joint bone; and guide mounting means to which the bone cutting guide is releasably mounted for releasably mounting the bone cutting guide to said joint bone,
the positioning guide including a bone mounting structure in the form of a moulding which is constructed from anatomical data obtained of said region of said joint bone so as to define complementary locating formations which correspond to anatomical formations of said region of said joint bone, thereby to provide for the secure fitment of the bone mounting structure to said region of said joint bone, the bone mounting structure having at least one cutting guide locating formation to which the bone cutting guide of the bone cutting guide assembly is releasably mounted, in use, for positioning the bone cutting guide, relative to said joint bone in an arrangement wherein the cutter guide formation of the bone cutting guide is located in a predetermined position relative to said joint bone so as to facilitate the cutting of said prosthetic joint locating face in said joint bone thereby to provide for the fitment of the prosthetic joint thereto.

2. The positioning guide as claimed in claim 1, wherein the cutting guide locating formation is in the form of a guide passage defined through the moulding.

3. The positioning guide as claimed in claim 1, wherein the cutting guide locating formation is in the form of an attachment post projecting from an external side of the moulding.

4. A bone cutting guide system for use in a joint replacement surgical procedure for guiding the cutting of at least one prosthetic joint locating face in a region of a predetermined joint bone of a human patient, from which a portion of bone is to be removed, thereby to allow for the secure fitment of a prosthetic joint to said bone in a predetermined orientation which approximates the anatomical normality of said joint bone, the bone cutting guide system including:
a bone cutting guide assembly including:
a) a bone cutting guide having at least one cutter guide formation for guiding a cutter for cutting said prosthetic joint locating face in said region of said bone; and
b) guide mounting means to which the bone cutting guide is releasably mounted for releasably mounting the bone cutting guide to said joint bone; and
the positioning guide as claimed in claim 1 for mounting the bone cutting guide assembly to said bone, with the bone cutting guide being fixedly secured to said bone after the mounting of the bone cutting guide to the cutting guide locating formation, with the cutting guide locating formation, the guide mounting means and the bone cutting guide being configured to permit removal of the guide mounting means and the positioning guide while the bone cutting guide remains fixedly secured to the bone, thereby to ensure the undisturbed attachment of the bone cutting guide to the bone at the predetermined position relative to the bone when the positioning guide and the guide mounting means are removed from the bone.

5. The bone cutting guide system as claimed in claim 4, wherein the bone cutting guide is in the form of an elongate base pin having a penetrating end configured to be imbedded into the bone, in use.

6. The bone cutting guide system as claimed in claim 5, wherein the cutting guide locating formation of the positioning guide, is in the form of an attachment post extending from an external side of the moulding.

7. The bone cutting guide system as claimed in claim 6, wherein the guide mounting means of the bone cutting guide assembly is in the form of a pin placement guide for guiding the placement of the base pin into the patient's bone, the pin placement guide having a mounting formation for mounting the pin placement guide to the attachment post and at least one pin mounting formation for guiding the insertion of the elongate base pin into the patient's bone.

8. The bone cutting guide system as claimed in claim 7, wherein the attachment post and the mounting formation of the pin placement guide define complementary configurations so as to permit the pin placement guide to be rotatably mounted to the attachment post, to permit the pin mounting formation of the pin placement guide to revolve around the attachment post to permit the selection of the most appropriate location for inserting the base pin into the patient's bone.

9. The bone cutting guide system as claimed in claim 7, wherein the attachment post and the mounting formation of the pin placement guide both have an angular configuration thereby to permit the pin placement guide to be mounted to the attachment post in a predetermined orientation, thereby to fix the position at which the pin mounting formation of the pin placement guide is located when the attachment post mounting formation of the pin placement guide is mounted to the attachment post.
